(19) United States
(12) Patent Application Publication
(10) Pub. No.: US 2002/0134385 A1
(43) Pub. Date: Sep. 26, 2002

(54) Regulator for underwater breathing devices

(76) Inventor: Nino Pietrelli, Sori (IT)

Correspondence Address: LARSON & TAYLOR, PLC, 1199 NORTH FAIRFAX STREET, SUITE 900, ALEXANDRIA, VA, 22314, US

(21) Appl. No.: 10/095,436

(22) Filed: Mar. 13, 2002

(30) Foreign Application Priority Data
Mar. 23, 2001 (IT) GE2001 A 000029

(51) Int. Cl. A62B 18/10; A62B 7/04; A61M 16/00; F16K 31/26

(52) U.S. Cl. 128/204.26; 128/201.28

(57) ABSTRACT

Regulator for underwater breathing devices including a relatively hard material box-like body having a pipe for the connection with a mouthpiece, at least one air inlet duct and at least one opening on which it is positioned one air exhaust valve; in said regulator the box-like body is externally covered by at least one layer of a suitably shaped relatively soft material.

### BACKGROUND AND SUMMARY OF THE INVENTION

The present invention refers to the regulators for underwater breathing devices.

As well-known the body of the regulators for underwater breathing devices include a box-like member, made generally from a comparatively hard material, provided with a pipe for the connection to a mouthpiece, a duct for the inlet of the air from a source of compressed air, such as diving bottles, and an opening on which it is positioned an air exhaust valve.

To the box-like body of the regulator are connected exhaust ducts (or sleeves) made of a material different from the one of the box-like body and whose main task is to channel the air exhausted from the said exhaust valve. In U.S. Pat. No. 4,784,129 a regulator is described in which said exhaust ducts are made of the same material of the box-like body of the regulator and integral with it, so to reduce the costs and the regulator maintenance problems, and to avoid that said exhaust ducts can disconnect and get lost during the diving. However said box-like body and the integral exhaust ducts are normally made using a relatively hard material, such as fiberglass or the like material, which proves to be discomforting, troublesome and little comfortable for the diver. Moreover said box-like bodies have standard shapes and colors, then turning out to be little personalizable by the diver.

The object of the present invention is to avoid the drawbacks of the known regulators by providing a regulator for underwater breathing devices comprising a box-like body made of relatively hard material and having a pipe for the connection with a mouthpiece, at least one air inlet duct and at least one opening on which it is positioned one air exhaust valve; said box-like body being externally covered by at least one layer of a relatively soft material suitably shaped so as to let uncovered the outer end of the mouthpiece pipe, the outer end of the compressed air inlet duct and to let the access to the air exhaust valve.

According to a further aspect of the present invention, said regulator includes a baffle of relatively soft material fastened to the covering layer of the box-like body and comprising exhaust ducts for the air coming out of the said exhaust valve.

Advantageously, the soft material, from which the baffle is made and the material forming the covering of the box-like body of the regulator, will be much more comfortable and easeful than the hard material used in the known prior art regulators. Moreover the diver will be able to choose the preferred color and shape for the covering layer of the box-like body of the regulator and for the baffle, said layer and said baffle being applied, through for instance a molding process, after the manufacture of the box-like body.

### BRIEF DESCRIPTION OF THE DRAWINGS

Further objects and advantages of the present invention will be better understood from the following description of one preferred embodiment of the invention, to be considered as a not limitative example and made with reference to the enclosed drawings where:

FIG. 1 is a perspective view of a known regulator for underwater breathing devices, without the exhaust duct which normally is mechanically assembled in a subsequent time.

FIG. 2 is a perspective view of a regulator for underwater breathing devices provided with the soft covering according to the present invention.

### DESCRIPTION OF THE PREFERRED EMBODIMENT OF THE INVENTION

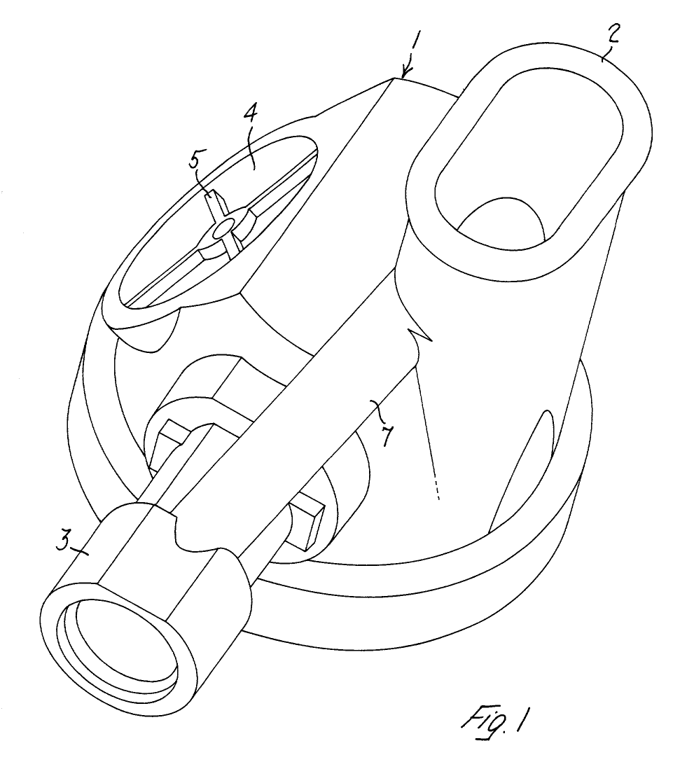

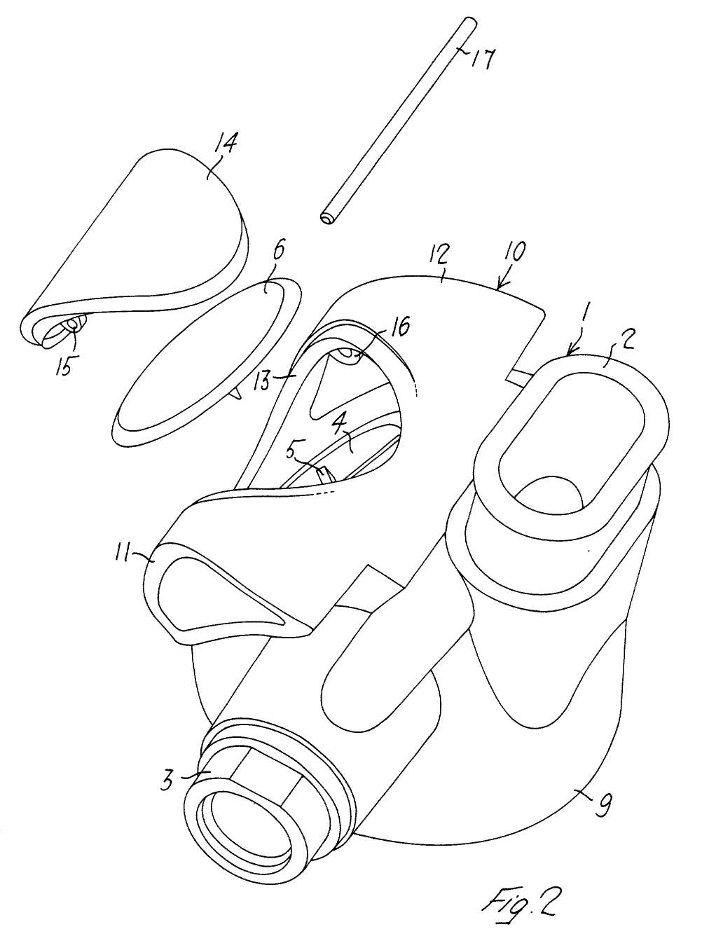

In FIG. 1 a regulator for underwater breathing devices is shown, formed by a box-like body 1 including a pipe 2 for the connection to a mouthpiece (not shown), a duct 3 for the inlet of the air from a source of compressed air, an opening 4 having a support for an exhaust valve 6 (shown in FIG. 2) and a duct 7 for the by-pass of the air from the compressed air inlet duct 3 to the connecting pipe 2 of the mouthpiece.

In FIG. 2 is shown the box-like body 1 to which it is applied, through for instance a molding process, a layer 9 of soft material. As it can be noticed, the layer 9 is shaped so to let an end part of the pipe 2 free for the following connection of the mouthpiece, to let free the end part of the inlet duct 3 for further elements connecting the compressed air source and finally to let unchanged the air exhaust opening 4. On this covering layer, on the air exhaust opening 4, is over-molded a baffle 10 made as well of a soft material, the same or different from the one of the covering layer 9. Said baffle 10 includes sideways two air exhaust ducts 11 and 12 and upwards an hole 13 provided with a removable closing cap 14, fit to allow the access and the eventual removal of the exhaust valve 6. On the inner layer of the cap 14 are provided two supporting rings 15 for an arm 17, said rings, when the cover 14 is positioned on the hole 13, aligned with two relative annular supports 16, each of them obtained by the side of the edge of said hole 13 and inside the upper surface of the baffle 10. The arm 17 then, to close said baffle 10, is inserted into the four holes of the rings 15 and of the annular supports 16.

Said baffle 10 can be, as mentioned before, made of the same or different soft material which the covering layer 9 is made of and moreover it can be changed in color, length and shape of the exhaust ducts 11 and 12 and other else as the diver using the present regulator likes. In case the material of the baffle 10 is the same of the layer 9, it will be fastened to the layer 9 at the same time of the application of said layer 9 to the regulator; in case said baffle 10 is made using a different material, it can be fastened to said layer 9 through suitable adhesive materials or other.

I claim:

1. In a regulator for underwater breathing devices including a box-like body made of relatively hard material and having a pipe for the connection with a mouthpiece, at least one air inlet duct and at least one opening on which it is positioned one air exhaust valve, the improvement according to which said box-like body is externally covered by at least one layer of a suitably shaped relatively soft material.

2. The regulator according to claim 1, in which said layer of relatively soft material is fastened to the said box-like body through over-molding of the soft material.

3. The regulator according to claim 1, further including a baffle element made preferably of the same material of the said covering layer of the box-like body and including ducts for the exhaust of the air coming out from said exhaust valve.

4. The regulator according to claim 3, in which said baffle is fastened to said covering layer through a over-molding process of the material which it is made of.

5. The regulator according to claim 1, in which said baffle is made of a material different from the one of the covering layer and it is fastened to this last through adhesion.

6. The regulator according to claim 3, in which said baffle includes two exhaust ducts positioned sideways as to said exhaust valve, said baffle being provided with an upper surface positioned in front of said valve and provided with at least one hole closed by a removable cap.

7. The regulator according to claim 6, in which said cap includes on its inner surface at least one supporting ring which, in the positioning of said cap onto the hole formed on the baffle, is aligned with at least two annular supports each one formed on the inner surface of said baffle and near the side edge of said hole, being said annular supports and said ring passed through by a removable arm.
